(19) United States
(12) Patent Application Publication
(10) Pub. No.: US 2016/0317486 A1
(43) Pub. Date: Nov. 3, 2016

(54) METHOD FOR WOUND HEALING

(71) Applicant: LEO Laboratories Limited, Dublin (IE)

(72) Inventors: Steven Martin Ogbourne, Pinbarren (AU); David Thomas, Cardiff (GB); Ryan Moseley, Maesteg (GB)

(21) Appl. No.: 15/131,851

(22) Filed: Apr. 18, 2016

Related U.S. Application Data
(63) Continuation of application No. 12/094,713, filed on Oct. 7, 2008, now abandoned.

(30) Foreign Application Priority Data
Nov. 25, 2005 (AU) 2005906601

(51) Int. Cl. A61K 31/22

(52) U.S. Cl. CPC A61K 31/22

(57) ABSTRACT

The present invention relates generally to methods and compositions for promoting wound healing in a subject. In particular, the invention relates to the use of ingenol compounds, particularly ingenol angelates, in wound healing and compositions thereof which contain such compounds.

### FIELD OF THE INVENTION

The present invention relates generally to methods and compositions for promoting wound healing in a subject. In particular, the invention relates to the use of ingenol compounds, particularly ingenol angelates, in wound healing and compositions therefor which contain such compounds.

### BACKGROUND OF THE INVENTION

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Wounds are external or internal injuries caused by inter alia, mechanical, chemical, thermal or pathogenic means which result in the physical disruption of structural tissue integrity.

Wound healing, i.e. the restoration of tissue (particularly cutaneous tissue) integrity, is orchestrated by various growth factors and cytokines which regulate cell growth, cell migration, cell differentiation and cell proliferation (Werner and Grose, 2003; Bryan et al, 2005). It can conveniently be described as occurring in three phases: (i) inflammation, (ii) proliferation and (iii) maturation, each of which can be further sub-catagorized into more specific stages; although none of these phases correspond to a precisely defined period of time and may overlap to some extent (Baum and Arpey, 2005). Numerous factors are involved in the complex process of wound healing following injury and cytokines are considered to play a key role in the regulation of the entire process (Hubner and Werner, 1996).

(i) Inflammatory (0-6 Days)

The first stage immediately following the infliction of the wound, such as a cutaneous wound, is referred to as hemostasis, whereby vasoconstriction and clotting, mediated by fibrin and platelets, are initiated to control bleeding. The clot further serves as a provisional matrix for incoming fibroblasts and inflammatory cells to the wound and as a reservoir of cytokines and growth factors.

Following hemostasis, inflammatory cells enter the wound and perpetuate the inflammatory process (manifested by erythema, heat, swelling and pain). The first of these are polymorphonuclear cells (PMNs) which are attracted by growth factors and cytokines such as platelet derived growth factor (PDOF) and IL-8. IL-8 is a major chemo-attractant for PMNs (Werner and Grose, 2003), and its rapid and transient expression is critical to the inflammatory process. PMNs begin to clean the wound by removing cellular debris, foreign particles and bacteria and are resident in the wound for a relatively short period (1-2 days). In turn, PMNs are a major source of cytokines such as IL-1α, IL-1β, IL-6 and TNF-α. By about 3 days post-injury, PMNs are replaced by monocytes, which transform into macrophages that also act as wound cleaners and a further source of IL-1α, IL-1β, IL-6 and TNF-α but tend to remain at the wound site for a longer period. IL-1β, IL-6 and TNF-α expression is strongly upregulated during the inflammatory phase (Grellner et al, 2000; Grose et al, 2002, Hubner et al, 1996) and their coordinate expression is likely to be important for normal repair (Hubner et al, 1996). Fibrocytes play an important role in the inflammatory process and are specifically involved in collagen and cytokine production, in part they are regulated by IL-1β and TNF-α.

(ii) Proliferation (3 Days—Several Weeks)

Granulation is an important bridging phase from inflammation to proliferation. Granulation tissue formation begins some 3-4 days after injury and primarily contains fibroblasts and macrophages. Migrating fibroblasts produce a permanent collagen-based extra-cellular matrix (ECM) and macrophages produce a variety of growth factors and cytokines such as IL-1 and TNF-α, which in turn stimulate the production of growth factors.

It has been demonstrated that fibroblast phenotype has a significant influence on both wound healing responses and clinical outcomes (Stephens et al, 1996, 2001, 2004). Studies have shown that fibroblasts from tissues which exhibit preferential wound healing in vivo (i.e. oral mucosal tissue) exhibit distinct phenotype responses in vitro (al-Khateeb et al, 1997). Furthermore, matrix metalloproteinases and serine proteinases play an important role in the regulation of cellular migration and ECM remodelling following injury and it has been demonstrated that decreased ECM reorganization and wound healing (i.e. chronic wounds) is associated with decreased fibroblast MMP production and activation (Cook et al, 2000). Oral mucosal and fetal skin fibroblasts demonstrate increased type I collagen lattice reorganization and contraction, associated with the superior capabilities of these cell types to migrate through the ECM and to repopulate experimental wound models in vitro, compared to normal skin fibroblasts (Stephens et al, 1996; al-Khateeb et al, 1997; Enoch, 2006). In contrast, chronic wounds fibroblasts are associated with decreased type I collagen lattice reorganization and contraction, associated with delayed or impaired cellular ECM migration and wound repopulation capabilities in vitro, compared to normal skin fibroblasts (Cook et al, 2000; Stephens et al, 2003; Wall, 2006). Increased MMP-2 levels and activity are associated with fibroblasts from oral musocal and fetal skin wound sites, whilst chronic wound fibroblasts have decreased MMP-2 levels and activity.

Re-epithelialization is the next key event in the wound healing process and is initiated primarily by migrating keratinocytes. Re-epithelialization is achieved via growth factor and cytokine stimulated proliferation of keratinocytes, which migrate through the granulation tissue. These cells appear to undergo a number of phenotypic changes during migration, expressing proteins associated with the differentiating cellular phenotype. As migration proceeds, keratinocytes acquire a proteolytic phenotype producing serine proteinases and MMPs. The keratinocytes continue to migrate into the wound space until completion, when the mitotically active keratinocytes undergo further phenotypic alteration, such that differentiation and stratification of the epithelium and re-formation of the basement membrane occurs, to complete the re-epithelialization process.

Cellular ECM attachment, ECM degradation by proteinases and the overall regulation of these processes by cytokines and growth factors, are other key features of wound remodelling and healing, which co-ordinate cellular function, such as cell migration and wound contraction, via cellular integrin-ECM interactions. Such interactions regulate cytoskeleton reorganization and new integrin-ECM interactions, via Rho family and actin binding proteins, whilst proteinases remove existing integrin interactions, allowing rear de-adhesion and cell migration (Martin, 1997: Stephens and Thomas, 2002; Kirfel et al, 2004). Cellular contractility in the absence of rear de-adhesion results in dermal reorganization, as quantified experimentally by collagen lattice reorganization/contraction.

IL-6 is considered to be crucial to “kick start” this aspect of the healing response (Werner & Grosse, 2003; Galluct et al, 2000) via its mitogenic effects on wound edge keratinocytes and its chemo-attractive effect on neutrophils. Transient expression of IL-6 is thought to be critical to scarless wound formation (Liechty et al, 2000).

(iii) Maturation (4 Days—Weeks or Months)

Wound maturation (or remodelling) may take as little as days or weeks but the complete process can last up to several years. During this phase contraction, decreased redness, decreased thickness, decreased induration and increased strength of the wound is observed. The wound contracts under the influence of myofibroblasts, collagen production in the granulation tissue decreases and blood vessels diminish. Wound healing is then completed by further re-epithelialization (Werner and Grose, 2003; Baum and Arpey, 2005).

Depending on the nature of the injury and the tissue, the disruption of the tissue integrity may render a subject vulnerable to infection, blood loss, loss of tissue function or scarring. Efficient and complete healing of a wound, is therefore vital for the continued health and well-being of the subject. Many factors can adversely affect the wound healing process, resulting in chronic or slow healing wounds, and/or scarring, and include the age and general health of the injured subject, malnutrition, diseases, applied pressure, impaired circulation, medication (such as anti-cancer and steroidal treatments), infection, the presence of foreign and necrotic tissue as well as the type of wound.

Furthermore, even once a wound has healed, scar tissue often remains. Scar tissue is both functionally and cosmetically inferior to normal uninjured skin. This inferiority is believed to be a consequence of the arrangement of collagen bundles within the neodermis generated during new tissue formation. The collagen bundles within normal skin are arranged in a complex 3-dimensional woven arrangement (often termed a “basket-weave” arrangement), which provides high levels of elasticity and resilience to damage, to the skin. Collagen bundles within scar tissue are arranged in a more planar manner, with bundles orientated parallel to the surface of the skin. The loss of 3-dimensional weave and its replacement with a parallel array of collagen bundles is believed to be responsible for the loss of cosmesis at sites of tissue scarring.

Promotion of wound healing remains the focus of intensive research and study and there are currently numerous methods and compositions available to treat wounds and promote wound healing, including a myriad of passive and active dressings and bandages, and topical medicaments, as well as physical and/or chemical debridement of necrotic tissue. Wound healing might also involve necrosis, apoptosis and alteration of the cell growth of non-transformed tissue.

Despite this, results have been somewhat inconsistent and the treatment of chronic or slow healing wounds continues to pose a serious challenge for the medical fraternity. There remains, therefore, the need for further agents and methods for treating wounds and the promotion of wound healing. Furthermore, agents capable of modulating the tissue repair process, in such a way as to promote the development of a more normal collagen architecture, would be expected to improve scar tissue quality.

The Euphorbiaceae family of plants covers a wide variety of plants including weeds of Euphorbia species. It is widely reported that a variety of ingenanes, particularly ingenol compounds are isolable from these species. One intensively studied species of this group is Euphorbia pilulifera L (synonyms E. hirta L., E. capitata Lam.), whose common names include pill-bearing spurge, snakeweed, cat's hair, Queensland asthma weed and flowery-headed spurge. The plant is widely distributed in tropical countries, including India, and in Northern Australia, including Queensland. Euphorbia peplus is another species from which ingenol angelates with anti-cancer properties have been isolated (See U.S. Pat. Nos. 6,432,452, 6,787,161 and 6,844,013). Ingenol-3-angelate is an ingenol angelate extracted and purified from E. peplus, and is useful, inter alia in the treatment of actinic keratoses and non-melanoma skin cancer (NMSC) by short term topical administration. The cytotoxicity of ingenol-3-angelate has been shown for many cell lines in vitro and its efficacy in vivo has been clinically established.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Given the critical roles performed by growth factors and cytokines as well as fibroblasts and keratinocytes in the process of wound healing, agents which may respectively modulate their production or phenotype response may be useful in treating wounds by promoting, stimulating, initiating, enhancing or otherwise progressing the wound healing process and/or reducing or minimizing scarring, i.e. improving cosmesis. It has now been shown that an ingenol compound can modulate immunostimulatory activity in peripheral blood mononuclear cell (PBMCs) and can up-regulate the expression or production of certain cytokines which play a role in wound healing. It has also been shown that the phenotype and pivotal wound healing responses of dermal fibroblasts and keratinocytes can be modulated using such a compound. Such modulated alterations may be beneficial to wound healing outcomes, particularly for cutaneous wounds. Advantageously, this may also result in wound healing outcomes with reduced scar formation. The present invention now provides new methods for modulating cytokine production and the phenotype response of fibroblasts and keratinocytes involved in wound healing. Thus, by stimulating the acute inflammatory response, such as increasing PMN and macrophage migration and increasing pro-inflammatory cytokine levels, wound healing can be promoted. Thus, the invention provides methods for wound healing and treating wounds. The invention also provides agents which promote the development of a more normal collagen architecture and thus may advantageously improve scar tissue quality of the healed wound.

Accordingly, in a first aspect, there is provided a method of modulating the production of one or more cytokines in a subject in need thereof, comprising administering to said subject a modulating effective amount of an ingenol compound or a pharmaceutically acceptable salt, or prodrug thereof.

In another aspect the invention provides a method of modulating the production of one or more cytokines at a wound site of a subject in need thereof, comprising administering to said subject a modulating effective amount of an ingenol compound or a pharmaceutically acceptable salt or prodrug thereof. In one embodiment, the administration involves topical application of the ingenol compound or a pharmaceutically acceptable salt, or prodrug thereof to the wound site.

In one embodiment, modulation involves increasing cytokine production.

In another embodiment, the one or more cytokines are selected from the group IL-1β, IL-2, IL6, IL-8 and TNF-α.

In another aspect, there is provided a method of modulating the phenotype response of dermal fibroblasts and/or keratinocytes in a subject in need thereof comprising administering to said subject a modulating effective amount of an ingenol compound or a pharmaceutically acceptable salt, or prodrug thereof.

In another aspect the invention provides a method of modulating the phenotype response of dermal fibroblasts and/or keratinocytes at a wound site of a subject in need thereof, comprising administering to said subject a modulating effective amount of an ingenol compound or a pharmaceutically acceptable salt or prodrug thereof. In one embodiment, the administration involves topical application of the ingenol compound or a pharmaceutically acceptable salt, or prodrug thereof to the wound site.

In another aspect, the present invention provides a method of promoting wound healing in a subject in need thereof, comprising administering to said subject a wound healing effective amount of an ingenol compound or a pharmaceutically acceptable salt, or prodrug thereof.

In a further aspect, the invention provides a method of treating a wound by promoting wound healing in a subject in need thereof, comprising topically applying a wound healing effective amount of an ingenol compound or a pharmaceutically acceptable salt, or prodrug thereof to the wound.

In one embodiment, the wound is a cutaneous wound such as a dermal or epidermal wound.

In some embodiments the ingenol compound is selected from ingenol-3-angleate, 20-O-acetyl-ingenol-3-angelate and 20-deoxy-ingenol-3-angelate and pharmaceutically acceptable salts and prodrugs thereof.

The compounds contemplated by the invention may desirably assist in restoring, developing or promoting normal collagen architecture and may therefore provide a method for reducing or minimizing scarring or otherwise improving the cosmetic or functional outcome, such as improved strength or elasticity, or reduced redness, thickness, induration, or hypo- or hyper-pigmentation of a wound. In doing so the compounds may provide an improved or accelerated rate for achieving this, particularly for chronic wounds whereby the inflammatory response may be “kick-started” to promote healing.

Therefore, in yet another aspect, the invention provides a method for reducing or minimizing scar tissue or improving cosmesis or functional outcome in a wound, comprising administering to the wound of a subject in need thereof a scar reducing or minimizing amount or cosmetic or functional improving amount of an ingenol angelate compound or a pharmaceutically acceptable salt or prodrug thereof.

### BRIEF DESCRIPTION OF THE FIGURES

FIG. 1 graphically depicts average tensiometric data obtained in acute (surgical), rat full-thickness incisional wounds, at (A) 4 wks and (B) 12 wks, following the application of 0.01%, 0.028%, 0.05% PEP005, compared with the DMSO/isopropanol vehicle (control) and untreated wound control groups. N-NT=PEP005-“naïve”, untreated; N-V=PEP005-“naïve”, vehicle-treated; V=PEP005-exposed, vehicle-treated.

### DETAILED DESCRIPTION OF THE INVENTION

Before describing the present invention in detail, it is to be understood that unless otherwise indicated, the subject invention is not limited to specific formulations of components, manufacturing methods, dosage regimes, or the like, as such may vary. It is also to be understood that the terminology used herein is for the purpose of describing particular embodiments only, and is not intended to be limiting.

The singular forms “a”, “an” and “the” include plural aspects unless the context clearly dictates otherwise. Thus, for example, reference to “an angeloyl substituted ingenane” or “an ingenol angelate” includes a single compound, as well as two or more compounds as appropriate.

As used herein, a “wound” refers to physical disruption of the continuity or integrity of tissue structure. “Wound healing” refers to the restoration of tissue integrity. It will be understood that this can refer to a partial or a full restoration of tissue integrity. Treatment of a wound thus refers to the promotion, improvement, progression, acceleration, or otherwise advancement of one or more stages or processes associated with the wound healing process.

The wound may be acute or chronic. Chronic wounds, including pressure sores, venous leg ulcers and diabetic foot ulcers, can simply be described as wounds that fail to heal. Whilst the exact molecular pathogenesis of chronic wounds is not fully understood, it is acknowledged to be multi-factorial. As the normal responses of resident and migratory cells during acute injury become impaired, these wounds are characterised by a prolonged inflammatory response, defective wound extracellular matrix (ECM) remodelling and a failure of re-epithelialisation.

The wound may be any internal wound, e.g. where the external structural integrity of the skin is maintained, such as in bruising or internal ulceration, or external wounds, particularly cutaneous wounds, and consequently the tissue may be any internal or external bodily tissue. In one embodiment the tissue is skin (such as human skin), i.e. the wound is a cutaneous wound, such as a dermal or epidermal wound.

The human skin is composed of two distinct layers, the epidermis and the dermis, below which lies the subcutaneous tissue. The primary functions of the skin are to provide protection to the internal organs and tissues from external trauma and pathogenic infection, sensation and thermoregulation.

The outermost layer of skin, the epidermis, is approximately 0.04 mm thick, is avascular, is comprised of four cell types (keratinocytes, melanocytes, Langerhans cells, and Merkel cells), and is stratified into several epithelial cell layers. The inner-most epithelial layer of the epidermis is the basement membrane, which is in direct contact with, and anchors the epidermis to, the dermis. All epithelial cell division occurring in skin takes place at the basement membrane. After cell division, the epithelial cells migrate towards the outer surface of the epidermis. During this migration, the cells undergo a process known as keratinization, whereby nuclei are lost and the cells are transformed into tough, flat, resistant non-living cells. Migration is completed when the cells reach the outermost epidermal structure, the stratum corneum, a dry, waterproof squamous cell layer which helps to prevent desiccation of the underlying tissue. This layer of dead epithelial cells is continuously being sloughed off and replaced by keratinized cells moving to the surface from the basement membrane. Because the epidermal epithelium is avascular, the basement membrane is dependent upon the dermis for its nutrient supply.

The dermis is a highly vascularized tissue layer supplying nutrients to the epidermis. In addition, the dermis contains nerve endings, lymphatics, collagen protein, and connective tissue. The dermis is approximately 0.5 mm thick and is composed predominantly of fibroblasts and macrophages. These cell types are largely responsible for the production and maintenance of collagen, the protein found in all animal connective tissue, including the skin. Collagen is primarily responsible for the skin's resilient, elastic nature. The subcutaneous tissue, found beneath the collagen-rich dermis, provides for skin mobility, insulation, calorie storage, and blood to the tissues above it.

Wounds can be classified in one of two general categories, partial thickness wounds or full thickness wounds. A partial thickness wound is limited to the epidermis and superficial dermis with no damage to the dermal blood vessels. A full thickness wound involves disruption of the dermis and extends to deeper tissue layers, involving disruption of the dermal blood vessels. The healing of the partial thickness wound occurs by simple regeneration of epithelial tissue. Wound healing in full thickness wounds is more complex. Cutaneous wounds contemplated by the invention may be either partial thickness or full thickness wounds.

Wounds contemplated by the invention include cuts and lacerations, surgical incisions or wounds, punctures, grazes, scratches, compression wounds, abrasions, friction wounds (e.g. nappy rash, friction blisters), decubitus ulcers (e.g. pressure or bed sores); thermal effect wounds (burns from cold and heat sources, either directly or through conduction, convection, or radiation, and electrical sources), chemical wounds (e.g. acid or alkali burns) or pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g. psoriasis and eczema, stomach or intestinal ulcers, oral wounds, including a ulcers of the mouth, damaged cartilage or bone, amputation wounds and corneal lesions.

Reference to an “ingenol” includes compounds having the C3, C4, C5-trioxy trans bicyclo[4.4.1]-undecane ingenane skeleton. Such compounds are extensively reported and known in the literature and can be isolated from plants such as from a species of the family Euphorbiaceae as well as chemically synthesized (see for example Winkler et al, 2002 and Tanino et al, 2003). The compounds are generally found in extracts of the Euphorbiaceae plants. An extract may comprise, therefore, sap or liquid or semi-liquid material exuded from, or present in, leaves, stem, flowers, seeds, bark or between the bark and the stem. Most preferably, the extract is from sap. Furthermore, the extract may comprise liquid or semi-liquid material located in fractions extracted from sap, leaves, stems, flowers, bark or other plant material of the Euphoriaceae plant. For example, plant material may be subject to physical manipulation to disrupt plant fibres and extracellular matrix material and inter- and intra-tissue extracted into a solvent including an aqueous environment. All such sources of the compounds are encompassed by the present invention including compounds obtained by chemically synthetic routes.

Reference herein to a member of the Euphorbiaceae family includes reference to species from the genera Acalypha, Acidoton, Actinostemon, Adella, Adenocline, Adenocrepts, Adenophaedra, Adisca, Agrostistachys, Alchornea, Alchorneopsis, Alcinaeanthus, Alcoceria, Aleurites, Amanoa, Andrachne, Angostyles, Anlsophyllum, Antidesma, Aphora, Aporosa, Aporosella, Argythamnla. Astrococcus, Astrogyne, Baccanrea, Baliospermum, Bernardia, Beyeriopsis. Bischofia, Blachia, Blumeodondron, Bonania, Bradlela, Breynla, Breyniopsis, Briedelia, Buraeavia, Caperonia, Caryodendron, Cellanella, Cephalocroton, Chaenotheca, Chaetocarpus, Chamaesyce, Cheilosa, Chiropetalum, Choriophyllum, Clcca, Chaoxylon, Cleidon, Cleistanthus, Cluytia, Cnesmone, Cnidoscolus, Coccoceras, Codiaeum, Coelodtscus, Conami, Conceveiba, Conceveibastrum, Concevetbum, Corythea, Croizatia, Croton, Crotonopsis, Crozophora, Cubanthus, Cumuria, Dactylostemon, Dalechampla, Dendrocousinsia, Diaspersus, Didymocistus, Dimorphocalyx Discocarpus, Ditaxis, Dodecastingma, Drypetes, Dysopsis, Elateriospermum, Endadenium, Endospermum, Erlsmanthus, Erythrocarpus, Erythrochilus, Eumecanthus, Euphorbia, Euphorbiodendron, Excoecaria, Flueggea, Calearia, Garcia, Gavarretta, Gelonium, Giara, Givotia, Glochidion, Clochidionopsis, Glycydendron, Gymnanthes, Gymnospara, Haematospermum. Hendecandra, Hevea, Hieronma Heronyma, Hippocrepandra, Homalanthus, Hymenocardia, Janipha, Jatropha, Julocroton, Lasiocroton, Lelocarpus, Leonardia, Lepidanthus, Leucocroton, Mabea, Macaranga, Mallotus, Manihot, Mappa, Maprounea, Melanthesa, Mercurialis, Mettenia, Micrandra, Microdesmis, Microelus, Microstachy, Maocroton, Monadenium, Mozinna, Neoscortechinia, Omalanthus, Omphalea, Ophellantha, Orbicularia, Ostodes, Oxydectes, Palenga, Pantadenia, Paradrypeptes, Pausandra, Pedilanthus, Pera, Peridium, Petalostigma, Phyllanthus, Picrodendro, Pierardia, Pilinophytum, Pimeleodendron, Piranhea, Platygyna, Plukenetla, Podocalyx, Poinsettia, Poraresia, Prosartema, Pseudanthus, Pycnocoma, Quadrasla, Reverchonia, Richeria, Richeriella, Ricinella, Ricinocarpus, Rottlera, Sagotia, Sanwithia, Sapium, Savia Sclerocroton, Sebastiana, Securinega, Senefeldera, Seneflderopss, Serophyton, Siphonla, Spathiostemon, Spixia, Stillingia, Strophioblachia, Synadenium, Tetracoccus, Tetraplandra, Tetrorchidium, Thyrsanthera, Tithymalus, Tragela, Trewia, Trigonostemon, Tyria and Xylophylla.

A preferred genus and particularly suitable for the practice of the present invention is the genus Euphorbia. Particularly useful species of this genus include Euphorbia aaron-rossii, Euphorbia abbreviata, Euphorbia acuta, Euphorbia alatocauls, Euphorbia albicaulis, Euphorbia algomarginata, Euphorbia aliceae, Euphorbia alta, Euphorbia anacampseros, Euphorbia andromedae, Euphorbia angusta, Euphorbia anthonyi, Euphorbia antiguensis, Euphorbia apocynifolia, Euphorbia arabica, Euphorbia ariensis, Euphorbia arizonica, Euphorbia arkansana, Euphorbia arteagae, Euphorbia arundelana, Euphorbia astroites, Euphorbia atrococca, Euphorbia baselicis, Euphorbia batabanensls, Euphorbia bergeri, Euphorbia bermudiana, Euphorbia bicolor, Euphorbia biformis, Euphorbia bifurcata, Euphorbia blobata, Euphorbia biramensis, Euphorbia biuncialis, Euphorbia blepharostipula, Euphorbia blodgetti, Euphorbia boerhaavloides, Euphorbia boliviana, Euphorbia bracei, Euphorbia brachiata, Euphorbia brachycera, Euphorbia brandegee, Euphorbia brittonil, Euphorbia caesia, Euphorbia calcicola, Euphorbia campestris, Euphorbia candelabrum, Euphorbia capitellata, Euphorbia carmenensis, Euphorbia carunculata, Euphorbia cayensis, Euphorbia celastroides, Euphorbia chalicophila, Euphorbia chamaerrhodos, Euphorbia chamaesula, Euphorbia chiapensis, Euphorbia chiogenoides, Euphorbia cinerascens, Euphorbia clarionensis, Euphorbia collmae, Euphorbia colorata, Euphorbia commutata, Euphorbia consoquitlae, Euphorbia convolvuloldes, Euphorbia corallifera, Euphorbia creberrima, Euphorbia crenulata, Euphorbia cubensis, Euphorbia cuspidata, Euphorbia cymbiformis, Euphorbia darlingtonii, Euphorbia defollata, Euphorbia degeneri, Euphorbia deltoidea, Euphorbia dentata, Euphorbia depressa Euphorbia dictyosperma, Euphorbia diclyosperma, Euphorbia dioeca, Euphorbia discoidalis, Euphorbia dorsiventralis, Euphorbia drumondi, Euphorbia duclouxii, Euphorbia dussrii Euphorbia eanophylla. Euphorbia eggersli, Euphorbia eglandulosa, Euphorbia elata, Euphorbia enalla, Euphorbia eriogonoIdes, Euphorbia eriophylla, Euphorbia esculaeformis. Euphorbia espirituensis, Euphorbia esula, Euphorbia excisa, Euphorbia exclusa, Euphorbia exstlpitata, Euphorbia exstipulata, Euphorbia fendleri, Euphorbia filicaulis, Euphorbia flliformis, Euphorbia florida, Euphorbia fruticulosa, Euphorbia garber, Euphorbia gaumerii, Euphorbia gerardiana, Euphorbia geyerl, Euphorbia glyptosperma, Euphorbia gorgonis, Euphorbia graciltor, Euphorbia gracillima, Euphorbia gradyi, Euphorbia graminea, Euphorbia graminlea Euphorbia grisea, Euphorbia guadalajarana, Euphorbia guanarensis, Euphorbia gymnadenia, Euphorbia haematantha, Euphorbia hedyotoides, Euphorbia heldrichii, Euphorbia helenae, Euphorbia helleri, Euphorbia helwigli, Euphorbia henricksonii, Euphorbia heterophylla, Euphorbia hexagona, Euphorbia hexagonoides, Euphorbia hinkleyorum, Euphorbia hintonit, Euphorbia hirtula, Euphorbia hirta, Euphorbia hoovert, Euphorbia humistrata, Euphorbia hyperic folia, Euphorbia inundata. Euphorbia nvoluta, Euphorbia jaliscensis, Euphorbia jejuna, Euphorbia johnston, Euphorbia juttae, Euphorbia knuthii, Euphorbia lasiocarpa, Euphorbia lata, Euphorbia latazti, Euphorbia latericolor, Euphorbia laxiflora Euphorbia lecheoides, Euphorbia ledienli, Euphorbia leucophylla, Euphorbia lineata, Euphorbia linguiformis, Euphorbia longecornuta, Euphorbia longepetiolata, Euphorbia longeramosa, Euphorbia longinsulicola, Euphorbia longipila, Euphorbia lupulina, Euphorbia lurida, Euphorbia lyctoides, Euphorbia macropodoides, macvaughiana, Euphorbia manca, Euphorbia mandonlana, Euphorbia mangletd, Euphorbia mango, Euphorbia marylandica, Euphorbia mayana, Euphorbia melanadenia, Euphorbia melanocarpa, Euphorbia meridensis, Euphorbia mertonit, Euphorbia mexiae, Euphorbia microcephala, Euphorbia microclada, Euphorbia micromera, Euphorbia misella, Euphorbia xpissurica, Euphorbia montana, Euphorbia montereyana, Euphorbia multicaulis, Euphorbla multiformis, Euphorbia multinodis, Euphorbia multiseta, Euphorbia muscicola, Euphorbia neomexicana, Euphorbia nephradenta, Euphorbia niqueroana, Euphorbia oaxacana, Euphorbia occidentalis, Euphorbia odontodenia, Euphorbia olivacea, Euphorbia olowaluana, Euphorbia opthalmica, Euphorbia ovata, Euphorbia paclhypoda, Euphorbia pachyrhiza, Euphorbia padifolia, Euphorbia palmeri, Euphorbia paludicola, Euphorbia parciflora, Euphorbia parishii, Euphorbia parryi, Euphorbia paxiana, Euphorbia pediculifera, Euphorbia peplidion, Euphorbia peploides, Euphorbia peplus, Euphorbia pergamena, Euphorbia perlignea, Euphorbia petaloidea, Euphorbia petaloidea, Euphorbia petrina, Euphorbia picachensis, Euphorbia pilosula, Euphorbia pilulifera, Euphorbia pinariona, Euphorbia pinetorum, Euphorbia pionosperma, Euphorbia platysperma, Euphorbia plicata, Euphorbia poeppigil, Euphorbia poliosperma, Euphorbia polycarpa, Euphorbia polycnemoides, Euphorbia polyphylla, Euphorbia portoricensis, Euphorbia portulacoldes Euphorbia portulana, Euphorbia preslil, Euphorbia prostrata, Euphorbia pteroneura, Euphorbia pycnanthema, Euphorbia ramosa, Euphorbia rapulum, Euphorbia remyi, Euphorbia retroscabra, Euphorbia revoluta, Euphorbia rivularis, Euphorbia robusta, Euphorbia romosa, Euphorbia rubida, Euphorbia rubrosperma, Euphorbia rupicola, Euphorbia sanmartensis, Euphorbia saxatilis M. Bieb, Euphorbia schizoloba, Euphorbia sclerocyathium, Euphorbia scopulorum, Euphorbia sentils, Euphorbia serpyltfolla, Euphorbia serrula, Euphorbia settloba Engelm, Euphorbia sonorae, Euphorbia soobyi, Euphorbia sparsiflora, Euphorbia sphaerosperma, Euphorbia syphilitica, Euphorbia spruceana, Euphorbia subcoerulea, Euphorbia stellata, Euphorbia submammilar s, Euphorbia subpeltata, Euphorbia subpubens, Euphorbia subreniforme, Euphorbia subtrifoliata, Euphorbia succedanea, Euphorbia tamaulipasana, Euphorbia telephioides, Euphorbia tenuissima, Euphorbia tetrapora, Euphorbia tirucalli, Euphorbia tomentella, Euphorbia tomentosa, Euphorbia torralbasil, Euphorbia tovariensls, Euphorbia trachysperma, Euphorbia tricolor, Euphorbia troyana, Euphorbia tuerckhelmi, Euphorbia turczaninowfl, Euphorbia umbellulata, Euphorbia undulata, Euphorbia vermiformis, Euphorbia versicolor, Euphorbia villifera, Euphorbia violacea, Euphorbia whitei, Euphorbia xanti Engelm, Euphorbia xylopoda Oreenm., Euphorbia yayalesla Urb., Euphorbia yungasensis, Euphorbia zeravschanica and Euphorbia zinniiflora.

Particularly preferred species of the genus Synadenium include Synadentum grantil and Synadenium compactum.

Particularly preferred species of the genus Monadenium include Monadenium lugardae and Monadenium guentheri.

A preferred species of the genus Endadenium is Endadentum gossweilent.

Euphorbia peplus is particularly useful in the practice of the present invention in terms of providing a source of ingenol angelates. Reference herein to “Euphorbia peplus” or its abbreviation “E. peplus” includes various varieties, strains, lines, hybrids or derivatives of this plant as well as its botanical or horticultural relatives. Furthermore, the present invention may be practiced using a whole Euphorbiaceae plant or parts thereof including sap or seeds or other reproductive material may be used. Generally, for seeds or reproductive material to be used, a plant or plantlet is first required to be propagated.

Reference herein to a Euphorbiaceae plant, a Euphorbia species or E. peplus further encompasses genetically modified plants. Genetically modified plants include trangenic plants or plants in which a trait has been removed or where an endogenous gene sequence has been down-regulated, mutated or otherwise altered including the alteration or introduction of genetic material which exhibits a regulatory effect on a particular gene. Consequently, a plant which exhibits a character not naturally present in a Euphorbiaceae plant or a species of Euphorbia or in E. peplus is nevertheless encompassed by the present invention and is included within the scope of the above-mentioned terms.

In one embodiment of the invention, the ingenol compound has the formula:

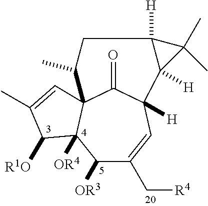

wherein
R¹-R³ are independently selected from hydrogen, optionally substituted alkyl, optionally substituted alkenyl, optionally substituted alkynyl, optionally substituted acyl, optionally substituted arylalkyl, S(O)₂R′, S(O)₂OR′, P(O)(OR′)₂ (wherein R′ is hydrogen, alkyl, alkenyl, alkynyl, acyl, aryl, or arylalkyl) and glycosyl; and R⁴ is selected from hydrogen, hydroxy, optionally substituted alkoxy, optionally substituted alkenoxy, optionally substituted alkynoxy, optionally substituted acyloxy, optionally substituted arylalkoxy, OS(O)₂R′, OS(O)₂OR′, OP(O)(OR′)₂ (wherein R′ is hydrogen, alkyl, alkenyl, alkynyl, acyl, aryl, or arylalkyl) and glycoxy.

In a one embodiment of the invention, at least one of R¹-R⁴ is not hydrogen. In a preferred form thereof, R¹ is not hydrogen.

In one particular embodiment of the invention, R¹ is an optionally substituted acyl group C(O)—R. In further embodiments thereof, R is optionally substituted alkyl, alkenyl or alkynyl. In a more preferred embodiment thereof, R may be straight chain or branched and may have up to 6 or up to 10 carbon atoms. In one embodiment thereof, R is branched.

In certain embodiments of the invention, one of R¹-R³ is an angeloyl group, as depicted by the formula below, or R⁴ is an O-angeloyl group. Such compounds are referred to herein as ingenol angelates. In a particularly preferred embodiment of the invention, R¹ is an angeloyl group.

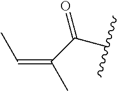 [SMILES: *C(=O)/C(C)=C\C]

In certain embodiments of the invention one or both of R² and R³ are hydrogen. R² and R³ may also form a methylene or ethylene dioxy group.

In certain embodiments of the invention R⁴ is hydrogen, hydroxy or acyloxy, such as acetoxy.

In certain embodiments of the invention, compounds for use in the described methods are ingenol-3-angelate, 20-O-acetyl-ingenol-3-angelate and 20-deoxy-ingenol-3-angelate and, pharmaceutically acceptable salts and prodrugs thereof.

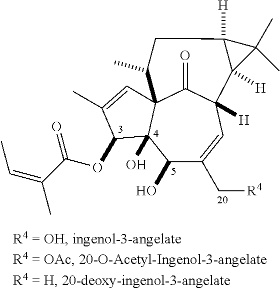

In a particular embodiment of the present invention the compound is ingenol-3-angelate (also referred to herein as “PEP005”). Reference herein to “ingenol-3-angelate” or “PEP005” includes naturally occurring as well as chemically synthetic forms.

Alkylation, alkenylation, alkynylation, arylalkylation or acylation can be carried out on the ingenol compounds using methods known in the art of synthetic chemistry for alkylating, alkenylation, alkynylation, arylalkylating or acylating free hydroxy groups (see for example, Greene and Wutz, 1999; March, 5^{th} Edition; Larock, 1999; the entire contents of which are incorporated herein by reference). For example, hydroxy groups can be alkylated (or arylalkylated) using alkyl (or arylalkyl) halides, such as methyl iodide (or benzylbromide), or dialkyl sulfates, such as dimethyl or diethyl sulfate. Acylation can be effected by treatment with appropriate carboxylic acids, acid halides and acid anhydrides in the presence of a base or a coupling agent. Glycosidic formation may be effected chemically, for example, by reacting the ingenol compound with a protected sugar compound in which C-1 has been activated by halogenation for coupling with the hydroxyl or carboxyl groups and the sugar hydroxyl groups have been blocked by protecting groups. Alternatively, glycoside formation may be effected enzymatically using an appropriate glycosyltransferase such as UDP-galactose dependent galactocyltransferase and UDP-glucose dependent glycotransferase. Preferred C-1 linked saccharides area furanose or pyranose saccharide (sugar) substituent which is linked to the ingenol angelate structure through C-1 of the saccharide (conventional numbering) to form an acetyl linkage. Exemplary saccharide groups include reducing sugars such as glucose, ribose, arabinose, xylose, mannose and galactoses, each being linked to an oxygen atom of the ingenol compound.

Sulfate, sulfonate and phosphate groups can be prepared by method known in the art. Examples of R′ include hydrogen, C₁₋₆alkyl, phenyl and benzyl.

As used herein, the term “alkyl” denotes straight chain, branched or cyclic alkyl, preferably C₁₋₂₀ alkyl, e.g. C₁₋₁₀ or C₁₋₆. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2,-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyl-octyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl (also referred to as “cycloalkyl”) include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substitutents as herein defined.

The term “alkenyl” as used herein denotes groups formed from straight chain, branched or cyclic hydrocarbon residues containing at least one carbon to carbon double bond including ethylenically mono-, di- or poly-unsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substitutents as herein defined.

As used herein the term “alkynyl” denotes groups formed from straight chain, branched or cyclic hydrocarbon residues containing at least one carbon-carbon triple bond including ethynically mono-, di- or poly-unsaturated alkyl or cycloalkyl groups as previously defined. Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substitutents as herein defined.

The term “aryl” denotes any of single, polynuclear, conjugated and fused residues of aromatic hydrocarbon ring systems. Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may be optionally substituted by one or more optional substituents as herein defined.

The term “acyl” denotes a group C(O)—R, wherein R is a hydrogen, alkyl, alkenyl, alkynyl, arylalkyl or aryl residue. Examples of acyl include formyl, straight chain or branched alkanoyl (e.g. C₁₋₂₀) such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanyl; cycloalkylcarbonyl such as cyclopropylcarbonyl cyclobutylcarbonyl, cyclopentylcarbonyl and cyclohexylcarbonyl; straight chain or branched alkenoyl (e.g. C₂₋₂₀) such as angeloyl; and aroyl such as benzoyl, toluoyl and naphthoyl. The R residue may be optionally substituted as described herein.

An arylalkyl group is an alkyl group as defined herein, substituted by an aryl group as defined herein. In one embodiment, the alkyl group is terminally substituted by the aryl group. Examples of arylalkyl include phenylC₁-C₂₀alkyl such as benzyl, phenylethyl, phenylpropyl, phenylbutyl, phenylpentyl and phenylhexyl. One or both of the alkyl and aryl groups may be independently optionally substituted by one or more optional substituents as described herein.

Optional substitutents for alkyl, alkenyl, alkynyl, arylalkyl, aryl, and thus acyl, include: halo (chloro, bromo, iodo and fluoro), hydroxy, C₁₋₆ alkoxy, C₁₋₆alkyl, phenyl, nitro, halomethyl (e.g. tribromomethyl, trichloromethyl, trifluoromethyl), halomethoxy (e.g. trifluoromethoxy, tribromomethoxy), C(O)C₁₋₆alkyl, amino (NH₂), C₁₋₆alkylamino, (e.g. methylamino, ethylamino and propylamino) diC₁₋₆alkylamino (e.g. dimethylamino, diethylamino and dipropylamino), CO₂H, CO₂C₁₋₆ alkyl, thio (SH) and C₁₋₆-alkylthio. An optional substituent also includes the replacement of a CH₂ group by a carbonyl (C═O) group or may be a methylene or ethylene dioxy group.

It will be recognized that during synthetic or semisynthetic processes for the preparation of ingenol compounds contemplated by the present invention, it may be necessary or desirable to protect other functional groups which may be reactive or sensitive to the reaction or transformation conditions undertaken. Suitable protecting groups for such functional groups are known in the art and may be used in accordance with standard practice. As used herein, the term “protecting group”, refers to an introduced fucmtionality which temporarily renders a particular functional group inactive. Such protecting groups and methods for their installation and subsequent removal at an appropriate stage are well known (Greene and Wulz, 1999).

The present invention also relates to prodrugs of ingenol compounds. Any compound that is a prodrug of an ingenol compound is within the scope and spirit of the invention. The term “prodrug” is used in its broadest sense and encompasses those derivatives that are converted in vivo, either enzymatically or hydrolytically, to the compounds of the invention. Such derivatives would readily occur to those skilled in the art, and include, for example, compounds where a free hydroxy group is converted into an ester or anhydride. Procedures for acylating the compounds of the invention, for example to prepare ester prodrugs, are well known in the art and may include treatment of the compound with an appropriate carboxylic acid, anhydride or chloride in the presence of a suitable catalyst or base. Other conventional procedures for the selection and preparation of suitable prodrugs are known in the art and are described, for example, in WO 00/23419, Design of Prodrugs, Hans Bundgaard, Ed., Elsevier Science Publishers, 1985, and The Organic Chemistry of Drug Desig and Drug Action, Chapter 8, pp 352-401, Academic press, Inc., 1992, the contents of which are incorporated herein by reference.

Suitable pharmaceutically acceptable salts of compounds include, but are not limited to salts of pharmaceutically acceptable inorganic acids such as hydrochloric, sulphuric, phosphoric nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds of the invention may be in crystalline form either as the free compounds or as solvates (for example, of water, i.e. hydrates, or of common organic solvents such as alcohols) and it is intended that both forms are within the scope of the present invention. Methods of solvation are generally known within the art.

In one or more embodiments of the invention, the use of ingenol compounds in wound healing may advantageously promote or improve the rate, degree, extent or time taken for one or more of the healing phases. Ingenol compounds may also be useful in attaining improved cosmetic outcomes from healing wounds, e.g. a reduction in the level or extent of scarring, redness, skin marking, or pigmentation (hyper- or hypo pigmentation) which might otherwise be associated with healing of a wound. In certain embodiments ingenol compounds may be useful in a prophylactic sense, e.g. as an anti-wrinkle treatment.

Subjects which may be treated in accordance with the present invention include mammalian subjects: humans, primates, livestock animals (including cows, horses, sheep, pigs and goats), companion animals (including dogs, cats, rabbits, guinea pigs), and captive wild animals. Laboratory animals such as rabbits, mice, rats, guinea pigs and hamsters are also contemplated as they may provide a convenient test system. Non-mammalian species such as birds, amphibians and fish may also be contemplated in certain embodiments of the invention. A subject may also be referred to herein as an individual, patient, animal or recipient.

As used herein, “modulating” when used in reference to cytokine production refers, as appropriate, to an increase or decrease in cytokine production. In a preferred embodiment, this relates to an increased, up-regulated or enhanced cytokine expression or production.

When used in reference to dermal fibroblasts and/or keratinocytes, “modulating” refers to an alteration (increase or decrease as appropriate) in one or more phenotype responses such as cell viability and proliferation, cellular matrix attachment, ECM reorganization, MMP production, fibroblast differentiation, cell morphology and cell migration.

A modulating effective amount is an amount when applied or administered in accordance with a desired dosing regime which is sufficient to modulate, preferably up-regulate, the production of cytokines to a desired level.

A wound healing, cosmesis or functional outcome improving effective amount of an ingenol compound is an amount which when administered or applied in accordance with the desired dosing regime is sufficient to initiate, stimulate, enhance, augment, accelerate or otherwise promote one or more stages or processes for wound healing to the desired extent or achieve the desired cosmetic effect or functional outcome. Treatment of a wound refers to effecting initiation, stimulation, enhancement, augmentation, acceleration or promotion of one or more stages or processes for wound healing to achieve the desired outcome.

Suitable effective amounts (dosage) and dosing regimens can be determined by the attending physician and may depend on the particular tissue type and wound being treated, the nature and severity of the wound, i.e. whether partial or full thickness, chronic or acute, as well as the general age, and health of the subject. The ingenol compounds may be administered at a time deemed appropriate during the wound healing process. Thus, the ingenol compounds may be administered immediately or soon after the wound has occurred, and/or at any subsequent stage of the wound healing process to promote healing and/or reduce scarring and/or improve cosmesis. The compounds may also be administered to existing scar tissue to minimize or reduce, inter alia, scarring, redness, thickness and/or hyper- or hypo-pigmentation.

The active ingredient may be administered in a single dose or a series of doses. While it is possible for the active ingredient to be administered alone, it is preferable to present it as a composition, preferably as a pharmaceutical composition, with one or more pharmaceutically acceptable adjuvants. Thus, the present invention also relates to the use of an ingenol compound or a pharmaceutically acceptable salt, or prodrug thereof in the manufacture of a medicament for modulating cytokine production, modulating phenotype response of dermal fibroblasts and/or keratinocytes, promoting wound healing or reducing or minimizing scar tissue or improving cosmesis or functional outcome in a wound.

Wound healing medicaments or compositions may contain the ingenol angelate compound in an amount of from about 0.0001% to up to 100% by weight. In preferred embodiments, the composition contains the ingenol compound in an amount of from about 0.0001% to up to about 10% by weight, for example about 0.0005, 0.001, 0.0025, 0.005, 0.01, 0.025, 0.05, 0.075, 0.1, 0.125, 0.15, 0.2, 0.25 or 0.5% to about 0.5, 1.0, 2.5 or 5.0%. In one embodiment of the invention, the ingenol compound is ingenol-3-angelate present in an amount of about 0.001 to about 1%.

The ingenol compounds may be administered in any suitable form, either locally, e.g. by topical application to the wound or by injection into the wound, or systemically, such as oral, parenteral (including subcutaneous, intramuscular, intravenous and intradermal), nasal, inhalation, rectal or vaginal administration.

In a preferred embodiment of the invention the ingenol compounds are administered, i.e. applied, topically at, and optionally around, the site of the wound. The ingenol compounds may be topically applied in any suitable form including solutions, emulsions (oil-in-water, water-in-oil, aerosols or foams), ointments, pastes, lotions, powders, gels, hydrogels, hydrocolloids and creams. Suitable carriers or additives include mineral oil, propylene glycol, polyoxyethylene, polyoxypropylene, emulsifying wax, sorbitan monostearate, polysorbate 60, cetyl esters wax, cetearyl alcohol, 2-octyldodecanol, cyclodextrin, isopropyl alcohol, ethanol, benzyl alcohol and water. Alternatively, the ingenol compounds may be presented in the form of an active occlusive dressing, i.e. where the ingenol compound is impregnated or coated on a dressing such as bandages, gauzes, tapes, nets, adhesive plaster, films, membranes or patches.

In one embodiment of the invention, the ingenol compound is topically applied in the form of an isopropyl alcohol-based gel.

The formulation of compositions and dressings contemplated herein is well known to those skilled in the art, see for example, Remington's Pharmaceutical Sciences, 18^{th} Edition, Mack Publishing, 1990. Compositions may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. The carrier for compositions contemplated by the present invention must be pharmaceutically acceptable in the sense of being compatible with the other ingredients of the composition and not injurious to the subject. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

It will be understood that the invention may also be practised in conjunction with the use of other supplementary biologically or physiologically active agents. Thus, the methods and compositions described herein may be used in conjunction with other biologically or physiologically active agents such as antiviral agents, antibacterial agents, antifungal agents, vitamins, such as A, C, D and E and their esters, and/or additional wound healing agents, including a growth factors and cytokines, such as those described herein. These additional agents may be formulated into a composition or dressing together with the ingenol compound or administered separately.

The ingenol compounds may also be presented as implants which comprise a biocompatible polymeric coated, impregnated or otherwise bearing the ingenol compound.

The ingenol compounds may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid (e.g. mouth wash); gel, ointment or as an oil-in-water liquid emulsion or a water-in-oil liquid emulsion.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent), preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions for rectal administration may be presented as a suppository with a suitable base comprising, for example, cocoa butter, glycerin, gelatin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient. It should be understood that in addition to the active ingredients particularly mentioned above, the compositions of this invention may include other agents conventional in the art having regard to the type of composition in question, for example, binders, sweeteners, thickeners, flavouring agents disintegrating agents, coating agents, preservatives, lubricants, buffers, anti-oxidants and/or time delay agent

The compounds of the invention may also be presented for use in veterinary compositions. These may be prepared by any suitable means known in the art. Examples of such compositions include those adapted for:
(a) oral administration, external application (e.g. drenches including aqueous and non-aqueous solutions or suspensions), tablets, boluses, powders, granules, pellets for admixture with feedstuffs, pastes for application to the tongue;
(b) parenteral administration, e.g. subcutaneous, intramuscular or intravenous injection as a sterile solution or suspension;
(c) topical application e.g. creams, ointments, gels, lotions etc as described above.

The invention will now be described with reference to the following Examples which are provided for the purpose of illustrating certain embodiments of the invention and are not intended to limit the generality hereinbefore described.

### EXAMPLES

### Example 1

### Effect of PEP005 on Cytokine Production

### Example 1.1

### Cytokine Production by PEP005-Treated Human Cells

Confluent cultures of Me10538 cells, keratinocytes, fibroblasts and neutrophils were incubated for 6 h in the absence or presence of PEP005 (1-100 ng/ml). The supernatants were harvested and analyzed for the presence of the following cytokines; TNF-α, IL-6 and IL-8 using a multiplex detection kit (Biosource International, Nivelles, Belgium). The results are depicted in Table 1. Units of detected proteins are pg/ml.

TABLE 1.1
Induction of pro-inflammatory cytokines in human cells in vitro. Cells were incubated with the indicated
concentration of PEP005 for 6 h and the supernatants analyzed for the indicated cytokines.
(ND—not detectable. nt—not tested). Units of detected proteins are pg/ml.

| PEP005 | Keratinocytes | | | Fibroblasts | | | Melanoma | | | Neutrophils | | |
|---|---|---|---|---|---|---|---|---|---|---|---|---|
| ng/ml | IL-8 | TNFα | IL-6 | IL-8 | TNFα | IL-6 | IL-8 | TNFα | IL-6 | IL-8 | TNFα | IL-6 |
| 0 | 995 ± 48 | 8 ± 1 | ND | 20 ± 1 | ND | 76 ± 6 | 4 ± 0.3 | ND | ND | 644 ± 271 | ND | ND |
| 1 | 3910 ± 148 | 510 ± 26 | ND | 79 ± 3 | ND | 81 ± 4 | <2 | ND | ND | 7089 ± 1293 | ND | ND |
| 5 | 4775 ± 178 | 847 ± 37 | ND | 160 ± 14 | ND | 85 ± 4 | 210 ± 6 | ND | ND | nt | ND | ND |
| 10 | 3895 ± 198 | 498 ± 29 | ND | 215 ± 12 | ND | 141 ± 6 | 737 ± 26 | ND | ND | 2241 ± 684 | ND | ND |
| 100 | 2950 ± 108 | 335 ± 21 | ND | 239 ± 9 | ND | 205 ± 5 | 390 ± 18 | ND | ND | 617 ± 52 | ND | ND |

### Example 1.2

An isopropyl alcohol gel containing 0.05% PEP005 or a placebo gel was topically applied to patients with actinic keratosis lesions. Prior to and three months after application of the gel (active or placebo) the patients skin texture was clinically assessed. Three months after application of the gel (active or placebo) the patient's skin markings, skin hyperpigmentation and skin hypopigmentation was clinically assessed. The results are presented in Tables 1.2 and 1.3 which indicate the number or percentage of patients that showed improvement, worsening or no change to skin texture or presence or absence of skin marking, hyperpigmentation or hypopigmentation. The data indicated that application of 0.05% PEP005 gel (in comparison to placebo) improved skin texture. The data also indicated that application of 0.05% PEP005 gel (in comparison to placebo) reduced the number of patients with skin markings, three months after drug application. Furthermore, the data indicated that application of 0.05% PEP005 gel (in comparison to placebo) did not result in skin hyper- or hypo-pigmentation, three months after drug application.

TABLE 1.2
Table 1.2. Wound healing and cosmetic effect of 0.05% PEP005 Topical Gel in
a Phase IIa clinical trial in actinic keratosis (numbers of patients).

| | Skin Texture | | | Skin Marking | | Scarring | | Hypopigmentation | | Hyperpigmentation | |
|---|---|---|---|---|---|---|---|---|---|---|---|
| | Improved | Worsened | No Change | Absent | Present | Absent | Present | Absent | Present | Absent | Present |
| Placebo Gel | 5 | 0 | 7 | 1 | 11 | 12 | 0 | 12 | 0 | 12 | 0 |
| 0.05% PEP005 Topical Gel | 10 | 0 | 5 | 6 | 9 | 15 | 0 | 13 | 0 | 15 | 0 |

TABLE 1.3
Table 1.3. Wound healing and cosmetic effect of 0.05% PEP005 Topical Gel in a
Phase IIa clinical trial in actinic keratosis (percentage of patients).

| | Skin Texture | | | Skin Marking | | Scarring | | Hypopigmentation | | Hyperpigmentation | |
|---|---|---|---|---|---|---|---|---|---|---|---|
| | Improved | Worsened | No Change | Absent | Present | Absent | Present | Absent | Present | Absent | Present |
| Placebo Gel | 41.7% | 0.0% | 58.3% | 8.3% | 91.7% | 100.0% | 0.0% | 100.0% | 0.0% | 100.0% | 0.0% |
| 0.05% PEP005 Topical Gel | 66.7% | 0.0% | 33.3% | 40.0% | 60.0% | 100.0% | 0.0% | 100.0% | 0.0% | 100.0% | 0.0% |

### Example 1.3

### Material and Methods

Compounds

PEP005 was provided as a dry powder. A stock solution of 23.55 mM was prepared in DMSO and aliquots were stored at −20° C. An aliquot of the stock solution was thawed on the day of use and stored at room temperature prior to and during dosing. Intermediate dilution steps were carried out using DMEM cell culture medium.

Isolation of PBMC

For the isolation of PBMC freshly drawn human blood treated with Li-Heparin as an anti-coagulant was used. Cells were diluted with three volumes of CliniMACS PBS/EDTA Buffer (Miltenyi, Bergisch Oladbach), carefully layered over FicollPaque (Amersham Biosciences, Freiburg) in a conical tube and centrifuged at 400×g for 40 minutes at 20° C. in a swinging-bucket rotor without brake. The upper layer was aspirated, leaving the mononuclear cell layer undisturbed at the interphase. The interphase cells (lymphocytes, monocytes and thrombocytes) were carefully transferred into a new conical tube. The conical tube was filled with CliniMACS PBS/EDTA Buffer and centrifuged at 300×g for 10 minutes at 20° C. The supernatant was completely removed. For removal of platelets the cell pellet was resuspended in 50 ml of Buffer and centrifuged at 200×g for 10 minutes at 20° C. The supernatant was completely removed and the last washing step was repeated. Cells were resuspended in DMEMMedium (Invitrogen, Karlsruhe) and counted in a Neubauer-hemocytometer.

Stimulation of PBMC

For the stimulation of PBMC 250.000 cells per well were seeded in a 96-well plate. PBMC of three different healthy donors were stimulated with PEP005 in three different concentrations (1, 10 and 100 nM) or LPS 1 μg/ml (Linaris, Wertheim-Bettingen), PMA 10 ng/ml (Sigma, Deisenhofen) and Ionomycin 1 μg/ml (Sigma, Deisenhofen), respectively. Cells were incubated at 37° C. and 5% C02 in a humidified atmosphere for 24 h.

Bead Suspension Assays

In a typical Bead Suspension Assay a cytokine is captured from a supernatant with bead bound antibodies. The cytokine is quantified with a secondary antibody to complete a sandwich immunoassay. Cytokine concentrations are calculated with the help of a standard curve for each cytokine. The cytokines IL-Iβ, IL-2, IL-6, IL-8 and TNF-α were quantitatively measured in the supernatant of the PBMC with a BioRad BioPlex System according to the manufacturer's instructions. All samples were measured in duplicates. All units of detected proteins are pg/ml.

Verification of Viability of PBMC

After removal of the cytokine containing supernatant the PBMC were tested for viability by flow cytometry. Propidium Iodide Staining Solution (0.1 μg/test of 1×10⁶ cells) was used to determine the amount of dead cells. Unstimulated PBMC were used for a negative control.

Results

Cytokine Production

To investigate the immunostimulating effects of PEP005, PBMCs from three different healthy donors were exposed for 24 h to PEP005 at concentrations of 1, 10 and 100 nM. The secretion of IL-1β, IL-2, IL-6, IL-8 and TNF-α into the supernatant was quantitatively measured by flow cytometry with the Bead Suspension Assays. The results are depicted in Tables 1.4-1.8.

TABLE 1.4
IL-1β production of PBMCs from donors GK, AW and HL
after incubation with PEP005 for 24 h at concentrations of 1, 10 and
100 nM. Units of detected IL-1β are pg/ml.

| | | | PEP005 | PEP005 |
|---|---|---|---|---|
| | vehicle control | PEP005 (1 nM) | (10 nM) | (100 nM) |
| Donor: GK | 0 | 94.49 | 61.62 | 0 |
| Donor: AW | 0 | 314.73 | 173.33 | 10.92 |
| Donor: HL | 0 | 125.17 | 98.04 | 11.76 |

TABLE 1.5
IL-2 production of PBMCs from donors GK, AW and HL after
incubation with PEP005B for 24 h at concentrations of 1, 10 and
100 nM. Units of detected IL-2 are pg/ml.

| | | | PEP005 | PEP005 |
|---|---|---|---|---|
| | vehicle control | PEP005 (1 nM) | (10 nM) | (100 nM) |
| Donor: GK | 0 | 82.68 | 60.3 | 10.56 |
| Donor: AW | 0 | 54.61 | 31.53 | 2 |
| Donor: HL | 0 | 17.86 | 19.47 | 12.84 |

An approximately 20 to 80-fold (mean: approximately 50-fold) increase of IL-2 levels in the supernatant of PBMCs from the three donors was observed at 1 nM PEP005.

TABLE 1.6
IL-6 production by PBMCs from donors GK, AW and HL after
incubation with PEP005B for 24 h at the concentrations 1, 10 and
100 nM. Units of detected IL-6 are pg/ml.

| | | | PEP005 | PEP005 |
|---|---|---|---|---|
| | vehicle control | PEP005 (1 nM) | (10 nM) | (100 nM) |
| Donor: GK | 68.69 | 320.61 | 216.09 | 0 |
| Donor: AW | 30.71 | 131.46 | 61.66 | 0 |
| Donor: HL | 11.88 | 69.48 | 73.97 | 95.43 |

PEP005 at 1 nM caused an approximately 4 to 6-fold increase of IL-6 levels in PBMC supernatants (almost 9-fold elevated IL-6 levels in PBMC supernatant).

TABLE 1.7
IL-8 production by PBMCs from donors GK, AW and HL after
incubation with PEP005 for 24 h at concentrations of 1, 10 and
100 nM. Units of detected IL-8 are pg/ml.

| | | | PEP005 | PEP005 |
|---|---|---|---|---|
| | vehicle control | PEP005 (1 nM) | (10 nM) | (100 nM) |
| Donor: GK | 4834.48 | 13652.6 | 9418.94 | 52.77 |
| Donor: AW | 7642.56 | 28029.68 | 11438.34 | 205.36 |
| Donor: HL | 2535.39 | 12148.42 | 18220.74 | 217.52 |

IL-8 levels in the supernatant of PBMCs were increased 3- to 5-fold, following exposure to PEP005 at 1 nM. Many different cells (e.g., monocytes/macrophages, T cells, neutrophils, fibroblasts, endothelial cells, keratinocytes, hepatocytes, astrocytes and chondrocytes) are capable of IL-8 production.

TABLE 1.8
TNF-α production by PBMCs from donors GK, AW and HL after
incubation with PEP005 for 24 h at concentrations of 1, 10 and
100 nM.

| | | | PEP005 | PEP005 |
|---|---|---|---|---|
| | vehicle control | PEP005 (1 nM) | (10 nM) | (100 nM) |
| Donor: GK | 0 | 148.42 | 76.14 | 19.44 |
| Donor: AW | 0 | 130.99 | 73.48 | 12.93 |
| Donor: HL | 0 | 90.72 | 71.6 | 35.75 |

High levels of the cytokine TNF-α were detected in the supernatants of PBMCs from all three donors, following incubation with PEP005. TNF-α levels ranged from approximately 120 nM (stimulation with PEP005 at 1 nM) to 70 nM (PEP005 at 10 nM) to 20 nM (PEP005 at 100 nM). No significant TNF-α levels were detected in the supernatant of PBMCs exposed to the vehicle only.

### Example 2

### Effect of PEP005 on Modulation of Phenotype and Wound Healing Responses of Dermal Fibroblasts and Keratinocytes

Materials And Methods

Dermal Fibroblast Cell Culture

A normal adult skin biopsy (6 mm) was obtained (n=1), with informed consent, from an individual attending the Oral Surgery Clinic, School of Dentistry, Wales College of Medicine, Cardiff. Following the application of a local anaesthetic, the dermal biopsy was collected and adult dermal fibroblast cultures established by single cell suspension technique, following enzymic degradation of the specimen. This technique has previously been reliably used to establish viable primary cultures of both oral and dermal fibroblasts in vitro (Cook et al, 2000; Stephens et al, 2001; 2003). Dermal fibroblasts were cultured in Fibroblast-Serum Containing Medium, containing Dulbecco's Modified Eagle's Medium (DMEM), supplemented with L-glutamine (2 mM), antibiotics (100 U/ml penicillin G sodium, 100 mg/ml streptomycin sulphate and 0.25 μg/ml amphotericin B) and 10% fetal calf serum (all purchased from Invitrogen Ltd., Paisley, U.K.). Dermal fibroblast cultures were maintained at 37° C., in a 5% CO₂/95% air atmosphere, with the culture medium being changed every 2-3 days. Dermal fibroblasts were used between passage 7-17, for all experiments.

Keratinocyte Cell Culture

Human, adult, epidermal keratinocytes, were purchased cryopreserved from Cascade Biologics Inc., Nottinghamshire, U.K. These cells (≧500,000 viable cells/vial) were tested to be >70% viable, with the capacity to proliferate for at least 16 population doublings. The epidermal keratinocytes were cultured in serum-free, EpiLife® Medium (Cascade Biologics Inc.), supplemented with antibiotics (100 U/ml penicillin G sodium, 100 mg/ml streptomycin sulphate and 0.25 μg/ml amphotericin B) and EpiLife® Defined Growth Supplement (EDOS, consisting of purified bovine serum albumin, purified bovine transferring, hydrocortisone, recombinant human insulin-like growth factor type-1, prostaglandin E2 and recombinant human epidermal growth factor, Cascade Biologics Inc.). Epidermal keratinocytes cultures were maintained at 37° C., in a 5% CO₂/95% air atmosphere, with the culture medium being changed every 2-3 days. Epidermal keratinocytes were used between 4-6 passages, for all experiments.

Preparation of PEP005

PEP005 was supplied by Peplin Limited, Brisbane, Australia, in 20 mg batches and stored at 4° C. When required, the PEP005 was solubilized in dimethyl sulphoxide (DMSO, >99.9%, Sigma Chemical Co., Dorset, U.K.), at a concentration of 10 mg/ml. The solution was mixed for 5 min or until the solution was clear and the PEP005/DMSO stock solution stored at 4° C., where stable for several months.

Prior to use, the PEP005/DMSO stock solution was removed from 4° C. storage and warmed to room temperature. The required volumes of PEP005/DMSO were aliquoted into a poly-propylene vessel and the PEP005/DMSO diluted to the required concentration (typically 0.01 μg/ml, 0.1 μg/ml, 1 μg/ml, 10 μg/ml and 100 μg/ml) in Fibroblast-Serum Containing Medium (for dermal fibroblast cultures) or serum-free, EpiLife® Medium (for epidermal keratinocyte cultures), with fresh PEP005/culture medium solutions being prepared daily, at the various concentrations above, due to solution stability. Prior to discarding PEP005/culture medium solutions, at least two volumes of 0.1% sodium hydroxide (Sigma Chemical Co.), in 95% ethanol/5% methanol (both from Fisher Scientific, Leicestershire, U.K.), was added to each solution, to decontaminate.

Assessment of Dermal Fibroblast/Keratinocyte Viability and Proliferation

The MTT [3-(4,5-dimethyl-2-thiazolyl)-2,5-diphenyltetrazolium bromide] dye-reduction assay was employed for the assessment of dermal fibroblast and epidermal keratinocyte cell viability and proliferation, according to Cook et al (2000). Following trypzinisation, dermal fibroblast or epidermal keratinocyte were seeded in 96-well microtitre plates (VWR International Ltd., Leicestershire, U.K.), at a cell density of 2.5×10³ cell/well and 5×10³ cell/well, respectively. Following cell seeding for 24 h and 48 h, respectively, the dermal fibroblast and epidermal keratinocyte culture medium were replaced with culture medium (100 μl/well), containing 0, 0.01 μg/ml, 0.1 μg/ml, 1 μg/ml, 10 μg/ml or 100 μg/ml PEP005 (six culture wells per PEP005 concentration). The dermal fibroblast and epidermal keratinocyte cultures were maintained at 37° C., in a 5% CO₂/95% air atmosphere, to 7 and 3 days respectively, with the respective PEP005-containing culture media, being changed every two days. Various controls (six culture wells per control) were also established in the 96-well microtitre plates at each time-point, including (i) dermal fibroblast and epidermal keratinocyte culture medium alone (cell-free), (ii) dermal fibroblast and epidermal keratinocyte in culture medium, containing 1% DMSO, (iii) dermal fibroblast and epidermal keratinocyte in culture medium, containing 0.1% DMSO, (iv) dermal fibroblast and epidermal keratinocyte in culture medium, containing 0.01% DMSO, and (v) dermal fibroblast and epidermal keratinocyte in culture medium, containing 0.001% DMSO.

At days, 1, 3, 5 and 7, sterile MTT (25 μl of a 5 mg/ml MTT solution in PBS, Sigma Chemical Co.) was added to the corresponding culture medium in each well and the 96-well microtitre plates maintained at 37° C., in a 5% CO₂/95% air atmosphere, for 4 h. Extraction buffer (100 μl), consisting of 10% sodium dodecyl sulphate (SDS, Sigma Chemical Co.) in 0.5 M N,N-dimethylformamide (Sigma Chemical Co.) was the added to each well and the 96-well microtitre plates maintained at 37° C., in a 5% CO₂/95% air atmosphere, for 4 h. The absorbance values of each well were read spectrophotometrically, using a Bio-Tek Instruments Microplate Autoreader EL311 (Fisher Scientific), at 540 nm. Each experiment was performed on three separate occasions.

Assessment of Dermal Fibroblast/Keratinocyte Extracellular Matrix Attachment

Dermal fibroblast and epidermal keratinocyte cellular attachment to type I collagen and to fibronectin, was performed according to Cook et al (2000) and Stephens et al (2004). The wells of 96-well microtitre plates were incubated at 4° C. overnight with 40 μg/ml rat-tail tendon type I collagen (Sigma Chemical Co.) or 40 μg/ml plasma fibronectin (Sigma Chemical Co.). Non-specific binding was blocked by incubation with 1% bovine serum albumin (Sigma Chemical Co.), at 4° C. for 4 h. Following trypsinization, cell suspensions (100 μl) of the dermal fibroblast or epidermal keratinocyte in serum-free culture medium, containing 0, 0.01 μg/ml, 0.1 μg/ml, 1 μg/ml, 10 μg/ml or 100 μg/ml PEP005 (six culture wells per PEP005 concentration), were both seeded into the 96-well microtitre plate wells, to a cell density of 2.5×10⁴ cell/well. The 96-well microtitre plates were maintained at 37° C., in a 5% CO₂/95% air atmosphere, for 1 h or 3 h, followed by the removal of non-adherent cells by aspiration. The remaining adherent dermal fibroblasts or epidermal keratinocytes were washed (×2) with PBS (100 μl), fixed in 70% ethanol (100 μl, Fisher Scientific) for 15 min and stained with 0.1% crystal violet solution (Sigma Chemical Co.), for 25 min. Excess crystal violet was removed by washing (×5) in double-distilled water, with the remaining stain being solubilized in 0.2% Triton X-100 solution (25 μl, Sigma Chemical Co.). Various controls (six culture wells per control) were also established in the 96-well microtitre plates at each time-point, including (i) dermal fibroblast and epidermal keratinocyte culture medium alone (cell-free), in the presence of type I collagen or fibronectin, (ii) dermal fibroblast and epidermal keratinocyte culture medium alone (cell-free), in the presence of bovine serum albumin, (iii) dermal fibroblast and epidermal keratinocyte culture medium alone (cell-free), in the presence of type I collagen/bovine serum albumin or fibronectin/bovine serum albumin, (iv) dermal fibroblast and epidermal keratinocyte in culture medium, in the presence of bovine serum albumin, (v) dermal fibroblast and epidermal keratinocyte in culture medium, containing 1% DMSO, in the presence and absence of type I collagen or fibronectin, and (vi) dermal fibroblast and epidermal keratinocyte in culture medium, containing 0.1% DMSO, in the presence and absence of type I collagen or fibronectin. The absorbance values of each well were read spectrophotometrically, using a Bio-Tek Instruments Microplate Autoreader EL311, at 540 nm. Each experiment was performed on three separate occasions, with the absorbance values obtained being expressed as an average for each group of samples.

Assessment of Dermal Fibroblast Extracellular Matrix Reorganization and Matrix Metalloproteinase Production

The ability of dermal fibroblasts to remodel/reorganize their ECM environment in the presence of PEP005 was examined by fibroblast populated collagen lattices (FPCLs), according to Cook et al (2000). Following trypsinization, dermal fibroblasts were suspended in Fibroblast-Serum Containing Medium, containing 10% gelatinase-free, fetal calf serum (prepared using a gelatin-A Sepharose column, GE Healthcare Ltd., Buckinghamshire, U.K.), to remove endogenous MMP-2 and MMP-9 activity. Dermal fibroblasts (5×10⁵ cells/750 μl gelatinase-free Fibroblast-Serum Containing Medium) were added to 53 mm bacteriological grade culture dishes (VWR International Ltd.), containing 3 ml 2×DMEM, gelatinase-free fetal calf serum (750 μl), 0.1 M sodium hydroxide (750 μl), 1.7 mg/ml rat-tail tendon type I collagen (225 0 μl, prepared according to Rowling et al, 1990) and PEP005 (0, 0.01 μg/ml, 0.1 μg/ml, 1 μg/ml, 10 μg/ml or 100 μg/ml PEP005), in a total volume of 7.5 ml (3 FPCLs per PEP005 concentration). Various controls (three FPCLs per control) were also established, including (i) Fibroblast-Serum Containing Medium alone (cell-free), and (ii) cells in Fibroblast-Serum Containing Medium, containing 1% DMSO. The FPCLs were maintained at 37° C., in a 5% CO₂/95% air atmosphere, for 1 h, for collagen polymerization to occur and the FPCLs detached from the plate edges and resuspended in 2 ml PEP005-free, Fibroblast-Serum Containing Medium, containing 10% gelatinase-free fetal calf serum. The FPCLs were maintained at 37° C., in a 5% CO₂/95% air atmosphere, for 14 days, with the culture medium being changed every day. The degree of ECM reorganization/lattice contraction was quantified from three separate lattice diameter measurements performed on each of the three replicate samples, at days 1, 2, 3, 4, 5, 6, 7, 10 and 14, after initial fabrication. FPCL conditioned medium, surrounding the lattices, was also collected from each individual FPCL, in the presence of 0, 0.01 μg/ml, 0.1 μg/ml, 1 μg/ml, 10 μg/ml or 100 μg/ml PEP005, for analysis of MMP production and activity at these time-points.

To determine the relative amounts of pro- and active MMP species produced by the cells in the FPCL systems, gelatin zymography was employed, according to Cook et al (2000). Equal volumes (15 μl) of FPCL conditioned medium were subjected to sodium dodecyl sulphate-polyacrylamide gel electrophoresis (SDS-PAGE), on pre-cast 10% gelatin zymography gels (Ready Gel 10% Gelatin Zymogram Gels, Bio-Rad Laboratories Ltd., Hertfordshire, U.K.), incorporated into a Mini-Protean 3 Gel Electrophoresis System (Bio-Rad Laboratories Ltd.), at 15 mA for 4-5 h. SDS was removed from the gels by soaking in 2.5% Triton X-100 solution (Sigma Chemical Co.), at room temperature, for 1 h. MMPs were activated by incubation in 25 mM Tris-HCl buffer, pH 7.6, containing 5 mM calcium chloride (Sigma Chemical Co.), 25 mM sodium chloride (Fisher Scientific) and 5% Brij 35 (Sigma Chemical Co.), at 37° C., overnight. Gels were stained with Coomassie Blue (0.05% Coomassie Blue, Sigma Chemical Co., in 12% acetic acid and 54% methanol, both Fisher Scientific), destained in 7.5% acetic acid and 5% methanol and the gel images captured using a GS-690 Imaging Densitometer and Image Analysis Software (Bio-Rad Laboratories Ltd.). MMP identification was confirmed by the appearance of clear bands at comparable molecular weights to an MMP-2 standard (Cook et al, 2000).

Each experiment was performed on two separate occasions, with the % reductions in lattice contraction and the MMP densitometric values obtained being expressed as an average for each group of samples

Assessment of Dermal Fibroblast Differentiation

The effects of PEP005 on dermal fibroblast differentiation to myofibroblasts, was examined by the extent of α-smooth muscle actin expression by the differentiating dermal fibroblasts, following stimulation with TGF-β. Following trypsinization, the dermal fibroblasts were suspended in Fibroblast-Serum Containing Medium, containing 10% fetal calf serum, at a cell density of 2.5×10⁴ cells/ml. Aliquots (250 μl/well) of the dermal fibroblast cell suspension were seeded into 8-well chamber slides (VWR International Ltd.) and maintained at 37° C., in a 5% CO₇/95% air atmosphere, until approximately 30-40% confluent. At this stage, the Fibroblast-Serum Containing Medium was replaced with culture medium (250 μl/well), containing 10 ng/ml TGF-β1 and 0, 0.01 μg/ml, 0.1 μg/ml, 1 μg/ml, 10 μg/ml or 100 μg/ml PEP005 (three chamber slide wells per PEP005 concentration). Various controls (three chamber slide per control) were also established, including (i) Fibroblast-Serum Containing Medium alone, (ii) cells in Fibroblast-Serum Containing Medium (cytokeratin or vimentin 1° Ab control), (iii) cells in Fibroblast-Serum Containing Medium, containing 10 ng/ml TGF-β and 1% DMSO, and (iv) cells in Fibroblast-Serum Containing Medium, containing 1% DMSO.

The chamber slides were maintained at 37° C., in a 5% CO₂/95% air atmosphere, for 3 days, by which time, the cells had reached approximately 75% confluence. Chamber slides were fixed in 1:1 ice cold, acetone:methanol (300 μl/well) for 20 min and blocked in 1% BSA in PBS, at 4° C., for 1 h. The chamber slides were washed (×2) in 0.1% BSA in PBS, and incubated with one of the following primary, (i) monoclonal, mouse anti-human α-smooth muscle actin primary antibody (1:30 in wash buffer, 250 μl/well, Sigma Chemical Co.), (ii) monoclonal, mouse anti-human cytokeratin IgG1 primary antibody (1:30 in wash buffer, 250 μl/well, DakoCytomation Ltd., Cambridgeshire, U.K.), or (iii) monoclonal, mouse anti-human vimentin IgG1 primary antibody (1:30 in wash buffer, 250 μl/well, DakoCytomation Ltd.). The chamber slides were incubated in primary antibody at room temperature, for 2 h, washed (×3) in 0.1% BSA in PBS, and incubated with polyclonal, rabbit anti-mouse IgG's, FITC conjugated, secondary antibody (1:50 in wash buffer, 250 μl/well, DakoCytomation Ltd.), at room temperature, for 1 h, avoiding light. The chamber slides were washed (×3) in 0.1% BSA in PBS, and the chambers removed for slide mounting with Vectashield® Mounting Medium (Vector Laboratories Ltd., Cambridgeshire, U.K.) and viewed by fluorescent microscopy (Leica Leitz Dialux 20EB fluorescent microscope, Leica Microsystems U.K. Ltd., Buckinghamshire, U.K.), with digital images being captured at a magnification of ×250. Each experiment was performed on two separate occasions.

Results

Assessment of Dermal Fibroblast/Keratinocyte Cell Viability and Proliferation

The average values obtained for dermal fibroblast cell inability/proliferation demonstrated that PEP005 has a cytotoxic effect on dermal fibroblasts keratinocytes at concentrations of 100 μg/ml, compared to untreated fibroblast controls.

However, at 10 μg/ml concentrations, PEP005 appeared to have a stimulatory effect at days 1, 3 and 5. Additionally, by day 7, 0.01 μg/ml and 0.1 μg/ml concentrations appeared to stimulate cell viability/proliferation.

Assessment of Dermal Fibroblast/Keratinocyte Extracellular Matrix Attachment

Epidermal keratinocyte attachment to type I collagen and plasma fibronectin demonstrated that PEP005 exhibited a significant dose-dependent stimulation of cell attachment to type I collagen, at 0.01-10 μg/ml concentrations. A similar trend towards a possible stimulation of epidermal keratinocyte attachment to plasma fibronectin was also apparent at 1-10 μg/ml.

Assessment of Dermal Fibroblast Extracellular Matrix Reorganization and Matrix Metalloproteinase Production

Type I collagen lattice contraction was significantly increased at 0.1 μg/ml PEP005. Pro and active MMP-2 levels were observed to increase at PEP005 concentrations of 0.01-0.1 μg/ml.

Assessment of Dermal Fibroblast Differentiation

Dermal fibroblasts, in the absence of TGF-β1, but in the presence of 1 μg/ml and 10 μg/ml PEP005 exhibited detectable α-smooth muscle actin microfilaments.

### Example 3

### Assessment of the Impact of PEP005 on Wound Healing Parameters

Materials and Methods

Preparation of PEP005

PEP005 was supplied by Peplin Limited, Brisbane, Australia as 0.01% (100 μg/ml), 0.028% (280 μg/ml) and 0.05% (500 μg/ml) preparations in a DMSO/isopropanol-based gel. PEP005-free, DMSO/isopropanol-based carrier gel was also supplied to serve as a vehicle control. The PEP005 and vehicle gels were stored at 4° C., where stable for several months.

Rat Incision Wound Healing Model

In order to examine the effects of PEP005 on the repair of acute (surgical) incisional wounds, involving minimal new tissue generation, the rat, full-thickness incisional wound healing model, was employed.

Animal Husbandry

Adult male Sprague Dawley rats (Harlan U.K. Ltd., Oxfordshire, U.K.), approximately 8-weeks old and weighing between 250-300 g, were used in this study. The animals were initially housed in groups of up to four per cage (cage dimensions 40×25×20 cm, with sawdust bedding, changed twice weekly), according to Home Office regulations, in an environment maintained at an ambient temperature of 23° C. with 12-hour light/dark cycles. The animals were provided food (Standard Rodent Diet) and water ad libitum. In order to acclimatize the animals to their surroundings, prior to experimentation, the animals were housed for a minimum of one week without disturbance, other than to refresh their bedding and to replenish their food and water provisions. Following wounding, animals were monitored under individual housed conditions until fully recovered from the procedure. Animals were then maintained individually for a period of 2 weeks (i.e. until their wounds have fully re-epithelialized). After this initial 2-week period, animals were maintained in groups of up to four for the remainder of the study. All animal procedures were performed in a Home Office licensed establishment, under U.K. Home Office Licenses (PPL: 40/2650; PIL: 70/4934 and PIL: 60/7661).

Creation of Full-Thickness Incisional Wounds

Previous anti-scarring studies (investigating cytokine inhibitors and neutralizing antibodies to cytokines) have tended to use multiple (incisional) wounds on a single animal, each wound in receipt of a different treatment. Due to its past use and acceptance, this multi-incisional wound model has been selected as the model of choice to evaluate the effects of PEP005 on full-thickness incisional wound healing, with treatments being rotated between animals, in order to allow for the known caudo-cranial differences in rodents.

Animals were anaesthetized using inhalation of Halothane and air, and the dorsum of each rat shaved and washed with the bactericide, chlorhexidine gluconate (0.05% aqueous). Four full-thickness incisional wounds (1 cm in length, include the panniculus carnosus and hypodermis) were created on the backs of each animal. Wounds remained unsutured (allowed to gape), in order to allow granulation tissue to form within the wounds. Following wounding (day 0), one of the four PEP005 concentrations (PEP005-free, DMSO/isopropanol gel vehicle, 0.01%, 0.028% or 0.05% PEP005) were applied to each wound, whilst non-treated wound controls remained untreated. Each animal group was maintained over each respective experimental/harvesting period, according to Table 2.1.

TABLE 2.1
Experimental groups established for 4 wks and 12 wks
(for wound tensile strength analysis and scar tissue
quality assessment).

| | | | Number of | |
|---|---|---|---|---|
| | | Group | Replicate Wounds | |
| Group | Treatment | Name | Week 4 | Week 12 |
| 1 | PEP005-Naive, | N-V | 10 | 12 |
| | Vehicle-Treated | | | |
| 2 | PEP005-Naive, | N-NT | 10 | 12 |
| | No Treatment | | | |
| 3 | PEP005-Exposed, | Vehicle | 10 | 10 |
| | Vehicle-Treated | | | |
| 4 | PEP005 [0.01%] | PEP-0.01 | 10 | 10 |
| 5 | PEP005 [0.028%] | PEP-0.028 | 10 | 10 |
| 6 | PEP005 [0.05%] | PEP-0.05 | 10 | 10 |

Application of PEP005 and Vehicle to Incisional Wound Sites

Immediately after injury, single treatments of the 0.01%, 0.028% PEP005, or DMSO/isopropanol vehicle gels, were applied at volumes of 10 μl per 100 mm² (1 μg/10 μl, 2.8 μg/10 μl and 5 μg/10 μl PEP005, respectively), to the marginal skin surrounding each wound, with a total area of 600 mm² of marginal skin receiving treatment. The gels were applied using a positive displacement pipette and spread evenly over the treatment area using a sterile spatula, with care being taken not to directly introduce preparations into wounds. Gels were allowed to dry for a period of 10 min, following application. In order to prevent animals from interfering with their wounds, each wound was dressed using dry sterile gauze (Release®, Johnson & Johnson Wound Management Ltd., North Yorkshire, U.K.) and secured with Millipore™ tape (3M UK plc, Berkshire, U.K.). Each animal was also fitted with an Elizabethan Collar, in order to prevent dressing removal. Dressings remained in place for a period of three days, post-wounding. Rats were maintained in their respective experimental groups for 1, 4 and 12 weeks, when the animals in each group were euthanized and the condition of wound and peri-wound tissues (in terms of viability, erythema, oedema, etc.), monitored at all assessment points, according to Table 2.1. Animals were also weighed during the course of the Study, to determine whether PEP005 exposure had any adverse effects on the general health/condition of the experimental animals.

Euthanasia, Tissue/Sample Harvesting and Processing

Harvesting (Weeks 4 and 12)

Wound tissue and normal marginal skin was excised and a single 3 mm strip incorporating the wound/scar removed from each wound, using a twin bladed instrument. Tissue strips were then stored in saline moistened surgical gauze (Topper, Johnson & Johnson Wound Management), at 4° C., prior to tensiometric analysis. The remaining wound tissue was fixed in 10% formalin, processed and embedded in paraffin wax. Transverse sections (6 μm) were taken and stained with both Haematoxylin and Eosin (for routine histological evaluation) and Mallory's stain (for matrix orientation analysis).

Tensiometric Assessment of Wound Strength (Week 4 and 12)

Wound strength increases with time after injury and is consequently a measure of wound maturity. Wound breaking strength was quantified using an Inatron Tensiometer (Instron Ltd., Buckinghamshire, U.K.), pre-calibrated to give full-scale readings of 5.0 kilograms force (kgf) for the tensiometric analysis of week 4 wounds and 50.0 kilograms force (kgf) for the tensiometric analysis of week 12 wounds. Tissue strips (3 mm) from each 0.01%, 0.028%, 0.05% PEP005. DMSO/isopropanol vehicle gel and untreated wound control group, were clamped into the grips of the Tensiometer, set to pull the margins of the wound apart at a “cross-head speed” of 50 mm/min. Breaking strength was measured as the maximal force necessary to cause separation of the wound margins.

Sear Tissue Quality Assessment (Weeks 4 And 12)

Matrix orientation was determined in histological specimens from each 0.01%, 0.028%, 0.05% PEP005, DMSO/isopropanol vehicle gel and untreated wound control group, by placing the sections onto a microscope stage and orientating/rotating the sections as to allow photomicrographs to be taken parallel to the surface of the skin. Digital images of each scar were then captured in the upper and mid scar regions. Representative areas of interest within each scar region were selected and the orientation of the matrix components within each area, measured using custom written image orientation software (CICA-MOS, Version 1.0), which generates data describing the directionality of collagen bundles within the histological specimen images, providing an output describing the orientation in 12×15° segments. Typically, normal skin tissue would possess limited horizontal directionality, with peaks in directionality at approximately 45° and 105°. In contrast, scar tissue would have a significant proportion of collagen bundles orientated close to the horizontal, with a very high level of directionality at 0-180° (i.e. planar, parallel to the surface of the skin), but very minimal directionality between 45 and 1200. Less sever scar tissue would possess more collagen bundles orientated in directions other than at 0-180°.

The present Study investigated two levels of tissue orientation, (i) scar tissue from each 0.01%, 0.028%, 0.05% PEP005 DMSO/isopropanol vehicle gel and untreated wound control group were compared in terms of the amounts of matrix, orientated parallel to the horizontal ±7.5° and (ii) in order to allow for possible errors in section orientation, prior to image capture, the possible impact of local cutaneous organelles (e.g. hair follicles), and undulations/irregularities in the skin surface, scar tissue from each group was also compared in terms of the amounts of matrix, orientated parallel from the horizontal, by ±22.5°. Ultimately, the greater the collagen bundle planar/horizontal directionality, the more severe the scarring.

Results

Tensiometric Assessment of Wound Strength

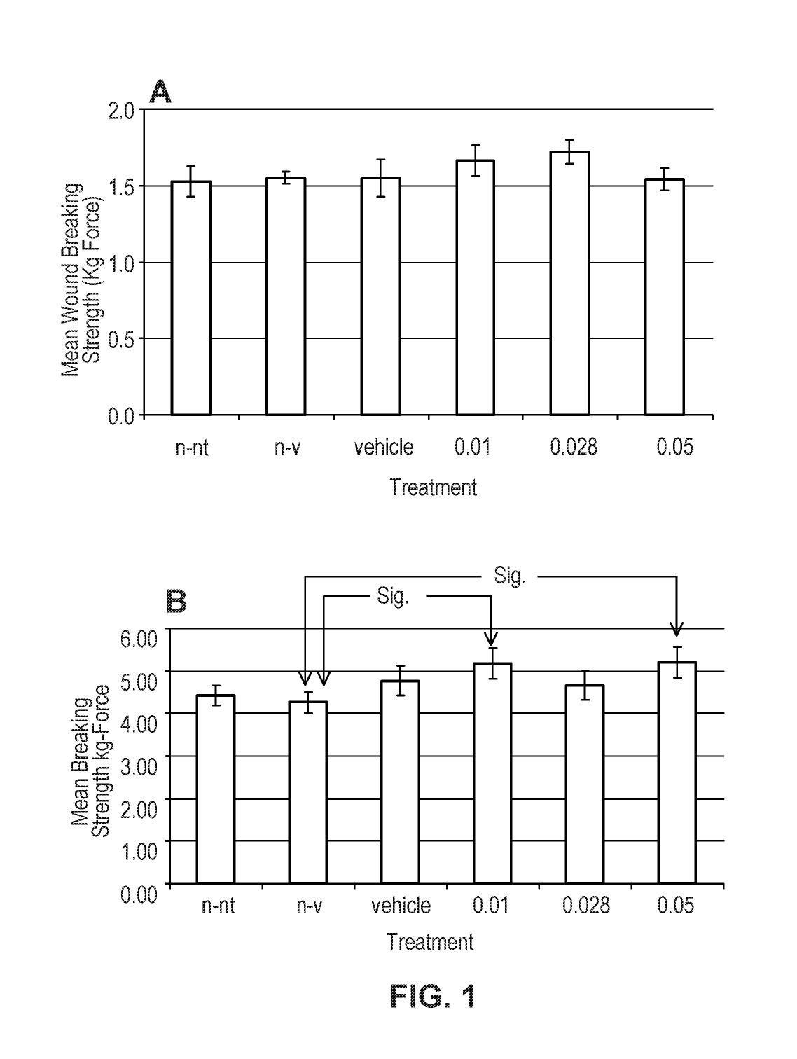

The average tensiometric, mean tensile strength values, obtained for tissue strips (3 mm) from each 0.01%, 0.028%, 0.05% PEP005, DMSO/isopropanol vehicle gel and untreated wound control group, at 4 weeks and 12 weeks, are shown in FIG. 1. The mean tensile strength values obtained at week 4 (FIG. 1A), demonstrated a dose dependent trend, with increasing tensile strength with PEP005 exposure.

The mean tensile strength values obtained at week 12 (FIG. 1B), demonstrated increased tensile strength values for all experimental groups, compared to week 4, with a biphasic trend following PEP005 treatment, increasing at 0.01%, declining at 0.028% to control levels, followed by another increase at 0.05% PEP005 concentrations.

Scar Tissue Quality Assessment

Topical treatment with 0.028% PEP005 reduced the percentage of collagen bundles that align at ±7.5° or ±22.5° compared to the three control groups.

Table 3.1 below provides average scar matrix orientation analysis data of the mid-wound displaying direction data at 17.5° to the horizontal and ±22.5° to the horizontal, in acute (surgical), rat full-thickness incisional wounds, following the application of 0.028% PEP005 compared with the DMSO/isopropanol vehicle (control) and untreated wound control groups at 12 weeks. N-NT=PEP005-“naïve”, untreated, N-V=PEP005=“naive”, vehicle treated; V=PEP005-exposed, vehicle treated.

TABLE 3.1

| | N-NT | N-V | V | 0.028% PEP005 |
|---|---|---|---|---|
| ±7.5° | 10.55% | 946% | 9.01% | 6.99% |
| ±22.5° | 30.32% | 26.84% | 26.03% | 21.81% |

### BIBLIOGRAPHY

al-Khateeb, T, Stephens, P. Shepherd, J P, Thomas, D W. An investigation of preferential fibroblast wound repopulation using a novel in vitro wound model. J Periodontol 1997; 68: 1063-1069.
Baum, C. L., Arpey, C. J., Normal Cutaneous Wound Healing: Clinical Correlation with Cellular and Molecular Events, Dermatol Surg 31:674-686 (2005). Bryan, D., Walker, K. B., Ferguson, M., Thorpe. R., Cytokine gene expression in a murine wound healing model, Cytokine 31:429-438 (2005).
Cook H, Stephens P, Davies J, Harding, K G, Thomas, D W. Defective extracellular matrix reorganization by chronic wound fibroblasts is associated with alterations in TIMP-1, TIMP-2, and MMP-2 activity. J Invest Dermatol 2000; 115: 225-33.
De Felici, M, Dolci, S. In vitro adhesion of mouse fetal germ cells to extracellular matrix components. Cell Differ Dev 1989; 26: 87-96.
Enoch, S. Phenotypic and genotypic characterisation of oral mucosal and patient-matched skin fibroblasts. PhD Thesis 2006; Wound Biology Group, Dept. Oral Surgery, Medicine & Pathology, Cardiff University.
Greene, T. W., and Wutz, P. G. M., Protective Groups in Organic Synthesis, Wiley InterScience, New York (1999).
Grellner, W., Georg, T., Wilske, J., Quantitative analysis of pro-inflammatory cytokines (IL-1beta, IL-6, TNF-alpha) in human skin wounds, Forensic Sci Int 113:251-264 (2000).
Grose, R., Werner S., Kessler, D., Tuckermann, J., Durka, S., Huggel, K., Reichardt, H., Werner, S. A role for endogenous glucocorticoids in wound repair, EMBO Reports 3:575-582 (2002).
Hübner, G., Brauchle, M., Smola, H., Madlener, M., Fassler, R., Werner, S., Differential regulation of pro-inflammatory cytokines during wound healing in normal and glucocorticoid-treated mice, Cytokine 8:548-556 (1996).
Kirfel, G, Rigort, A, Borm, B, Herzog, V., Cell migration: Mechanisms of rear detachment and the formation of migration tracks, Eur J Cell Biol 2004; 83: 717-724.
Larock, R. E., Comprehensive Organic Transformations, VCH Publishers (1999).
Liechty, K. W., Adzick, N. S. and Crombleholme, T. M., Diminished interleukin 6(IL-6) production during scarless human fetal wound repair, Cytokine 12:671-676 (2000).
March, J., Advanced Organic Chemistry, 5th Edition, Wiley InterScience, New York.
Martin, P., Wound healing—aiming for perfect skin regeneration, Science 1997; 276: 75-81.
Puschel, H U, Chang, J, Muller P K, Brinckmann, J., Attachment of intrinsically and extrinsically aged fibroblasts on collagen and fibronectin, J Photochem Photobiol 1995; 27: 39-46.
Stephens, P, Cook, H, Hilton, J, Jones, C J, Haughton, M F, Wyllie, F S, Skinner, J W, Harding, K G, Kipling, D, Thomas, D W., An analysis of replicative senescence in dermal fibroblasts derived from chronic leg wounds predicts that telomerase therapy would fail to reverse their disease specific cellular and proteolytic phenotype, Exp Cell Res 2003; 283: 22-35.
Stephens, P, Davies, K J, al-Khateeb, T, Shepherd, J P, Thomas, D W., A comparison of the ability of intra-oral and extra-oral fibroblasts to stimulate extracellular matrix reorganization in a model of wound contraction, J Dent Res 1996; 75: 1358-1364.
Stephens, P, Davies, K J, Occleston, N, Pleass, R D, Kon, C, Daniels, J, Khaw, P T, Thomas, D W., Skin and oral fibroblasts exhibit phenotypic differences in extracellular matrix reorganization and matrix metalloproteinase activity, Br J Dermatol 2001; 144: 229-237.
Stephens, P, Grenard, P, Aeschlimann, P, Langley. M, Blain, E, Errington, R, Kipling, D, Thomas, D W, Aeschlimann, D., Crosslinking and G-protein functions of transglutaminase 2 contribute differentially to fibroblast wound healing responses, J Cell Sci 2004; 117: 3389-3403.
Stephens P, Thomas D W., The cellular proliferative phase of wound healing, J Wound Care 2002; 11: 253-261.
Wall, I B., A cellular and molecular analysis of chronic wound and normal dermal fibroblasts, PhD Thesis 2006; Wound Biology Group, Dept. Oral Surgery, Medicine & Pathology, Cardiff University.
Werner, S., Grose, R., Regulation of Wound Healing by Growth Factors and Cytokines, Physiol Rev 83:835-870 (2003).

1. A method of promoting wound healing in a subject in need thereof: comprising administering to said subject a wound healing effective amount of an ingenol compound or a pharmaceutically acceptable salt thereof.

2. A method for reducing or minimizing scar tissue or improving cosmesis or functional outcome in a wound, comprising administering to the wound of a subject in need thereof a scar reducing or minimizing amount or cosmetic or functional improving amount of an ingenol compound or a pharmaceutically acceptable salt thereof.

3. A method of modulating the phenotype response of dermal fibroblasts and/or keratinocytes in a subject in need thereof, comprising administering to said subject a modulating effective amount of an ingenol compound or a pharmaceutically acceptable salt thereof.

4. A method of modulating the phenotype response of dermal fibroblasts and/or keratinocytes at a wound site of a subject in need thereof, comprising administering to said subject a modulating effective amount of an ingenol compound or a pharmaceutically acceptable salt thereof.

5. A method of modulating the production of one or more cytokines in a subject in need thereof, comprising administering to said subject a modulating effective amount of au ingenol compound or a pharmaceutically acceptable salt thereof.

6. A method of modulating the production of one or more cytokines at a wound site of a subject in need thereof, comprising administering to said subject a modulating effective amount of an ingenol compound or a pharmaceutically acceptable salt thereof.

7. The method according to claim 5 wherein one or more cytokines are selected from the group consisting of IL-Iβ, IL-2, IL6, IL-8 and TNF-α.

8. The method according to claim 1 wherein the ingenol compound or pharmaceutically acceptable salt thereof is applied topically to the wound.

9. The method according to claim 1 wherein the ingenol compound has the formula: 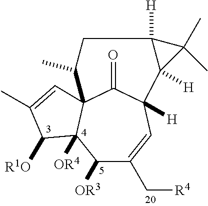
wherein
R¹-R³ are independently selected from hydrogen, optionally substituted alkyl, optionally substituted alkenyl, optionally substituted alkynyl, optionally substituted acyl, optionally substituted arylalkyl, S(O)₂R′, S(O)₂OR′, P(O)(O′)₂ (wherein R′ is hydrogen, alkyl, alkenyl, alkynyl, acyl, aryl, or arylalkyl) and glycosyl; and
R⁴ is selected from hydrogen, hydroxy, optionally substituted alkoxy, optionally substituted alkenoxy, optionally substituted alkynoxy, optionally substituted acyloxy. optionally substituted arylalkoxy, S(O)₂R′, OS(O)₂ OR′, OP(O)(OR′)₂ (wherein R′ is hydrogen, alkyl, alkenyl, alkynyl, acyl, aryl, or arylalkyl) and glycoxy.

10. The method according to claim 9 wherein the compound is selected from ingenol-3-angelate, 20-O-acetyl-ingenol-3-angelate and 20-deoxy-ingenol-3-angelate and, pharmaceutically acceptable salts thereof.

11. The method according to claim 10 wherein the compound is ingenol-3 angelate.

12. The method according to claim 1 wherein the wound is selected from the group consisting of cuts and lacerations, surgical incisions, punctures, grazes, scratches, compression wounds, abrasions, friction wounds, chronic wounds, ulcers, thermal effect wounds, chemical wounds, wounds resulting from pathogenic infections, skin graft/transplant donor and recipient sites, immune response conditions, oral wounds, stomach or intestinal wounds, damaged cartilage or bone, amputation sites and corneal lesions.

13. The method according to claim 12 wherein the wound is a cutaneous wound.

14. The method according to claim 12 wherein the wound is a chronic wound.

15. The method according to claim 14 wherein the wound is a diabetic-associated wound.
